(19) United States
(12) Patent Application Publication
(10) Pub. No.: US 2004/0077539 A1
(43) Pub. Date: Apr. 22, 2004

(54) $g(a)-lactalbumin as prebiotic agent

(76) Inventor: Kamiel Maase, Wagenheim (DE)

Correspondence Address: KEUSEY, TUTUNJIAN & BITETTO, P.C., 14 VANDERVENTER AVENUE, SUITE 128, PORT WASHINGTON, NY, 11050, US

(21) Appl. No.: 10/467,005

(22) PCT Filed: Feb. 1, 2002
(86) PCT No.: PCT/EP02/01241

(30) Foreign Application Priority Data
Feb. 1, 2001 (EP) 01200365.3

(51) Int. Cl. A61K 47/00; A61K 38/38

(52) U.S. Cl. 514/12; 424/439

(57) ABSTRACT

Described are the use of α-lactalbumin and α-lactalbumin enriched whey protein concentrate as a prebiotic food, food additive or food supplement, as well as methods for improving the prebiotic activity of ready-to-consume food products and such ready-to-consume food products including α-lactalbumin. Further, pharmaceutical compositions for the treatment of gastro-enteritis including α-lactalbumin as active compound are described.

### TECHNICAL FIELD OF THE INVENTION

The present invention relates to the use of α-lactalbumin and of α-lactalbumin enriched whey protein concentrate as a prebiotic food, food additive or food supplement, and to methods for improving the prebiotic activity of food products, as well as to ready-to-consume food products having improved prebiotic activity and to pharmaceutical compositions, comprising α-lactalbumin as active ingredient.

### DESCRIPTION OF RELATED ART

In the food industry, there is need for food products that may confer health benefits to the consumer by improving the beneficial intestinal microbial population, usually mainly consisting of the lactic acid producing bacteria of the lactobacilli, and bifidobacteria.

Two approaches for the preparation of such products are known in the art. The direct approach provides living preparations of the organisms in the food product, usually in fermented dairy products, such as yoghurt. Such beneficial organisms, present in food products are also known as “probiotics”. Nutritional preparations comprising probiotics are e.g. described in EP-A-904 784 and in WO-A-99/02170. One of the problems encountered with probiotics is that a substantial proportion thereof does not survive the passage of the gastro-intestinal tract upstream of the colon, the target location for such bacteria.

The above-mentioned problem is avoided by the so-called indirect approach, wherein food supplements are added to the food products, which food supplements support and stimulate the growth of the above-mentioned beneficial organisms resident in the gastrointestinal tract, in particular in the colon. These food additives are known as “prebiotics”. A wide range of non-digestible carbohydrates having prebiotic properties have been described. For example the use of fructo-oligosaccharides as prebiotic in natural dessert cream is described in EP-A-970 618; the use of D-tagatose as prebiotic is known from WO-A-9 943 217; inulin as prebiotic in a food preparation is described in WO-A-9 907 239, and β-glucans and derived compounds as prebiotics are described in WO-A-9 826 787. Further, fructan as prebiotic is known from U.S. Pat. No. 5,840,361.

### SUMMARY OF THE INVENTION

It has now surprisingly been found that in addition to the above-mentioned carbohydrates, the protein α-lactalbumin, a protein occurring in e.g. bovine milk, shows a significant prebiotic effect. Although it is known that whey can stimulate the growth of bifidobacteria in the colon (see e.g. B. A. Wharton et al. In: Lactoferrin: Structure and Function, T. W. Hutchnes et al (eds.), Plenum Press, New York, USA 1994; S. E. Balmer et al, Arch. Dis. Child. 1989: 1678-1684; B. Kleessen et al, Acta Peadiatr. 1995; 84: 1347-1356), the prebiotic effect thereof has been ascribed to the whey protein glycomacropeptide (GMP), a casein fragment being formed by the rennet in the curdling of milk in the cheese preparation process. It has now been found that α-lactalbumin and α-lactalbumin enriched whey protein concentrate have improved prebiotic properties compared to whole whey. The invention therefore relates to the novel use of α-lactalbumin, and also to the use of α-lactalbumin enriched whey protein concentrate as a prebiotic food, food ingredient, food additive or food supplement.

In one aspect of the present invention, use of α-lactalbumin as a prebiotic food, a food ingredient, a food additive or a food supplement is provided.

In another aspect, use of α-lactalbumin enriched whey protein concentrate as a prebiotic food, a food ingredient, a food additive or a food supplement is provided.

In yet another aspect, a method for improving prebiotic activity of a ready-to-consume food product is provided comprising the step of adding α-lactalbumin during the preparation of the said food product in such an amount that the α-lactalbumin content in the food product in ready-to-consume form is at least 0.5 w/w %.

In yet another aspect, a ready-to-consume food product is provided, comprising at least 0.5 w/w % α-lactalbumin.

In yet another aspect, use of α-lactalbumin as an active compound in the manufacture of a medicament for treatment of gastroenteritis is provided.

In yet another aspect, use of α-lactalbumin as an active compound in the manufacture of a medicament for enhancing a resident population of lactic acid producing micro-organisms in the gastrointestinal tract of an animal is provided.

In yet another aspect, a pharmaceutical composition for treatment of gastro-enteritis or for enhancing a resident population of lactic acid producing micro-organisms in the gastrointestinal tract of an animal, comprising α-lactalbumin and an iron compound as active ingredients, is provided.

These and other aspects, features and advantages of the present invention will be described or become apparent from the following detailed description of preferred embodiments, which is to be read in connection with the accompanying drawings.

### BRIEF DESCRIPTION OF THE DRAWINGS

The invention will now be further discussed by the following examples and figures, wherein:

FIG. 1 shows the effect of the presence of α-lactalbumin on the number of lactobacilli in rat faeces;

FIGS. 2A, 2B and 3 show the effect of casein, WPC and α-lactalbumin on the amount of lactobacilli in the rat caecum and colon

FIGS. 4A and 4B show growth of lactobacilli in faeces extracts from rats, fed with diets containing different protein preparations;

FIG. 5 shows a graph of the effect of the α-lactalbumin content in the rat diet on excretion of Salmonalla enteritidis;

### DETAILED DESCRIPTION OF PREFERRED EMBODIMENTS

The invention relates to the novel use of α-lactalbumin, and also to the use of α-lactalbumin enriched whey protein concentrate as a prebiotic food, food ingredient, food additive or food supplement.

When α-lactalbumin enriched whey protein concentrate is used, the α-lactalbumin content in the α-lactalbumin enriched whey protein concentrate is preferably at least 60 w/w %. When e.g. a whey protein concentrate having an α-lactalbumin content below 20% is added to a food product, in particular a dairy product, a relatively large amount thereof has to be added in order to obtain the desired α-lactalbumin level in the dairy product. In order to avoid possible negative organoleptic effects by adding a too high amount of protein to the food product, it is preferred to use protein concentrates, comprising at least 60 w/w % α-lactalbumin. With such concentrates, an α-lactalbumin content of 30 w/w % (on total protein basis) can be obtained in a ready-to-consume dairy product, e.g. yoghurt, without any negative organoleptic effects.

In a second aspect, the invention relates to a method for improving the prebiotic activity of a ready-to-consume food product, comprising the step of adding α-lactalbumin during the preparation of the said food product in such an amount that the α-lactalbumin content in the food product in ready-to-consume form is at least 0.5 w/w %, preferably 0.5-1.5 w/w %, more preferably about 1 w/w %. The term “about” allows deviation of 0.1%, preferably of 0.05 w/w %.

The terms “food product in ready-to-consume form”, “ready-to-consume food product” and “consumer food product” are to be understood as food products, that are suitable for direct consumption, without the necessity of substantially changing the composition thereof. Ready-to-consume food products may however be processed before consumption (without substantially altering the composition thereof), by e.g. whipping, (e.g. in the case of cream), or by freezing (e.g. in the case of ice cream formulations) or thawing (e.g. frozen foods) the food product.

Industrial food base materials, such as whey proteins, or whey protein concentrates, optionally enriched in α-lactalbumin content, are as such not consumable, but are used in the preparation of numerous food products, such as infant formulas and ice cream compositions. Such food base materials are therefore not considered as “ready-to-consume” food products. The term “whey protein concentrate” is known in the art; see e.g. Walstra et al, 1999, Dairy Technology, ISBN 0-8247-0228-X. Such an enriched concentrate, herein also referred to as “αWPC”, is known from EP-B-0 604 684. The α-lactalbumin content of αWPC may vary, depending on the preparation between 20-80 w/w %, whereas the α-lactalbumin content of normal WPC is about 12-18 w/w %. In this respect, reference is also made to EP-A-0 443 763, WO-A-01/05243, U.S. Pat. No. 4,485,040, U.S. Pat. No. 5,503,864 and FR-A-2 669 810. It is e.g. known from these references to incorporate αWPC in infant milk formulas.

According to the invention, the prebiotic activity of virtually any ready-to-consume food product can be improved; the skilled person will be aware of suitable ready-to-consume food products that can be produced according to the method of the present invention. Exemplary ready-to-consume food products are e.g. dairy products, drinks, soups, sauces, syrups, candy bars, nutritional bars, clinical and/or health food preparations, sports foods (e.g. in the form of tablets, capsules) and processed meat, poultry or fish products (such as sausages), as well as ready-to-consume meals, such as microwave-heatable pre-prepared meals.

“Improved prebiotic activity” is to be understood as the improvement of prebiotic activity of the food product as produced according to the method of the invention as compared to the same food product, without the addition of α-lactalbumin during the preparation process thereof. In this way, prebiotic activity can be conferred to food products that initially did not have prebiotic activity, or the prebiotic activity of food product having a certain degree of prebiotic activity can be further enhanced.

The α-lactalbumin can be added in any suitable processing step during the preparation of the food product; the skilled person will be aware of suitable steps or stages of the preparation process where the α-lactalbumin can be added. Also, the α-lactalbumin can be added as a last step of the food preparation process. α-Lactalbumin can be added in purified form, or e.g. as whey protein concentrate, that preferably be enriched in α-lactalbumin content. The α-lactalbumin can suitably be added in e.g. liquid form, e.g. as solution or suspension, or in the form of powder. According to the invention, α-lactalbumin can e.g. be incorporated in a formulation of a dry powder to be dissolved in water or another beverage for the preparation of instant drinks.

Many ready-to-consume food products comprising α-lactalbumin are known in the art. Bovine milk and yoghurt for example comprise about 0.1 w/w % α-lactalbumin; formulated milk for infants comprises about 0.1-0.3 w/w % α-lactalbumin. The said α-lactalbumin level is usually achieved by incorporating αWPC in the infant milk formula; however, the said formulations do not, when liquified to the proper envisaged concentrations mimicking real mother milk, have the high α-lactalbumin content as obtained according to the method according to the present invention.

It is now found that the above method results in food products having improved and beneficial prebiotic activity as compared with the corresponding food products, not comprising the additional α-lactalbumin.

In a preferred embodiment, the food product in ready-to-consume form is a dairy food product comprising casein, wherein the ratio α-lactalbumin:casein is at least 0.1, preferably between 0.1-0.6, preferably between 0.2-0.4. This means that on weight basis, the food product has a casein content that is at most 10 times as much as the α-lactalbumin content. Dairy products usually comprise both casein and α-lactalbumin. However, the ratio of α-lactalbumin: casein in dairy products is 0.07 or less. Thus, a dairy product, produced according to the method of the invention having α-lactalbumin:casein ratio of at least 0.1 is enriched in α-lactalbumin. Because of the elevated α-lactalbumin level in the dairy product, the dairy product shows a significant and improved prebiotic effect.

Preferably, the α-lactalbumin:casein ratio is 0.1-0.6. Above the value of 0.6, the organoleptic properties of the dairy product may become less attractive. Most preferably, the said weight ratio is 0.2-0.4; within these limits, significant prebiotic effects can be obtained, without interfering with the organoleptic qualities of the dairy product.

According to the invention, α-lactalbumin may be incorporated in any ready-to-consume food product; however dairy products, especially milk and yoghurt are preferred. With “dairy products” all ready-to-consume products, derived from milk are meant, such as milk, cheese, yoghurt, cream etc. Industrial food base materials such as casein and whey protein concentrate are as such not regarded as consumer products. The dairy ready-to-consume product is preferably chosen from milk, fermented milk products, such as yoghurt, instant drinks, desserts, cheese and cheese products, most preferably being chosen from milk and fermented milk products, such as yoghurt, fresh cheese, soft curd cheese, kefir, buttermilk, and in a very advantageous embodiment the dairy product is a yoghurt or yoghurt product, such as mild yoghurt of e.g. the ABT-type.

In a preferred embodiment of the method of the invention, at least a portion, but preferably all, of the α-lactalbumin is added as α-lactalbumin enriched whey protein concentrate (αWPC), for reasons as explained above. The αWPC preferably has a α-lactalbumin content of at least 60 w/w %, in order to avoid significant organoleptic consequences in the food product, as explained above.

In an attractive embodiment, the method of the invention further comprises the step of adding during the preparation of the said food product a food grade iron compound. The skilled person will be aware of suitable food grade iron compounds for incorporation in the food product. The skilled person will further be able to find the proper amount of such an iron compound to incorporate in the food product. Reference is made to Wharton et al., supra, herein incorporated by reference. It has namely been found that the combination of α-lactalbumin with the presence of a food grade and biocompatible iron compound has a synergistic effect on the prebiotic activity, compared to that of α-lactalbumin in absence of such a compound.

The invention also relates to a ready-to-consume food product, comprising at least 0.5 w/w %, preferably 0.5-1.5 w/w %, more preferably about 1 w/w % α-lactalbumin. In a preferred embodiment, the food product is a dairy product comprising casein, enriched in α-lactalbumin, wherein the ratio of α-lactalbumin:casein is at least 0.1, preferably between 0.1-0.6, more preferably between 0.2-0.4. Preferably, the ready-to-consume food product is a dairy product, preferably chosen from milk, fermented milk products, desserts, instant drinks, cheese and cheese products, preferably chosen from milk and fermented milk products; in a very special embodiment, the dairy product is yoghurt. In yet another embodiment, the ready-to-consume food product comprises a food grade iron compound.

Preferably, the protein part of the food product according to the invention comprises 10-40, preferably 20-35, most preferably 25-30 w/w % α-lactalbumin.

Further, the invention relates to the use of α-lactalbumin as active compound in the manufacture of a medicament for treatment of gastro-enteritis and for enhancing a resident population of lactic acid producing micro-organisms in the gastrointestinal tract of an animal, preferably a mammal, including humans. Herein, the term “gastro-enteritis” is meant to encompass both gastritis and enteritis and simultaneous gastritis and enteritis in both acute and chronic forms. As α-lactalbumin has been shown to be active as prebiotic agent, the said compound is very suitable for incorporation in a pharmaceutical composition against intestinal diseases, especially gastro-enteritis and for enhancing a resident population of lactic acid producing micro-organisms in the gastrointestinal tract of an animal. Any suitable administration form can be chosen for the pharmaceutical composition such as tablets, syrups, suppositories etc. For the preparation of the pharmaceutical composition, α-lactalbumin can be combined with any suitable carrier, diluent, excipient or any other suitable additives known in the art. It is to be noted that the use of α-lactalbumin, because of the high tryptophan content therein, in a medicament for controlling the circadian rhythm, usable against a disrupted sleeping rhythm, is described in FR-A-2 754 181.

Preferably, the α-lactalbumin content in the medicament is 0.2 w/w % or more, preferably 0.4 w/w % or more, most preferably 0.6 w/w % or more.

The invention also relates to a pharmaceutical composition, in particular for treatment of gastro-enteritis or for enhancing a resident population of lactic acid producing micro-organisms in the gastrointestinal tract of an animal, comprising as active ingredient 0.2 w/w % or more, preferably 0.4 w/w % or more, most preferably 0.6 w/w % or more α-lactalbumin. Preferably, the pharmaceutical composition comprises both α-lactalbumin and an iron compound as active ingredients. In view of the envisaged aim, the skilled person will be able to select a proper iron compound, as well as the amount of such an iron compound to incorporate in the pharmaceutical composition.

### EXAMPLES

### Example 1

Effect of α-Lactalbumin on the Total Lactobacillus Counts in Rat Faeces

Male Wistar rats, SPF (specific pathogen free), six weeks old, were divided into groups as is depicted in Table 1. Animals were fed a diet containing 10% protein, 20% fat (50% palm oil, 50% corn oil), 63% glucose, 2% cellulose, and 5% of a vitamin and mineral mixture according to AIN 93. Water was supplied ad libidum.

TABLE 1

| Group | Rat Numbers | Protein source | g α-lac./100 g diet |
|---|---|---|---|
| A | 1, 4, 7, 10, 13, 16 | Casein | 0.00 |
| B | 2, 5, 8, 11, 14, 17 | WPC 80* | 0.48 |
| C | 3, 6, 9, 12, 15, 18 | αWPC** | 2.40 |

*WPC 80 (Esprion 580, DMV International, The Netherlands). This product contains approximately 12% α-lactalbumin/protein
**α-lactalbumin enriched WPC 80 (60% α-lactalbumin/protein)

In the “casein” diet (A), the protein source was 100% casein.

In the “WPC” diet (B), the protein consisted of 50% casein and 50% WPC.

In the “WPC” diet (C), the protein consisted of 50% casein and 50% α-WPC.

After 14 days, the amount of lactobacilli (LAB) was determined in the faeces. The total amount of lactobacilli is determined by plating dilutions of fresh faeces on MRS plates (see: J. C. de Man et al, Appl. Bact. 1960; 23: 130-135, purchased from E. Merck Nederland B. V., Amsterdam, The Netherlands). The composition of the lactobacilli flora was determined by analysing a representative sample of the colonies on the MRS plates by RAPD-PCR, followed by 16S RNA sequence analysis (H. J. M. Harmsen et al, J. Pediatr. Gastroenterol, Nutr. 2000; 30: 61-67).

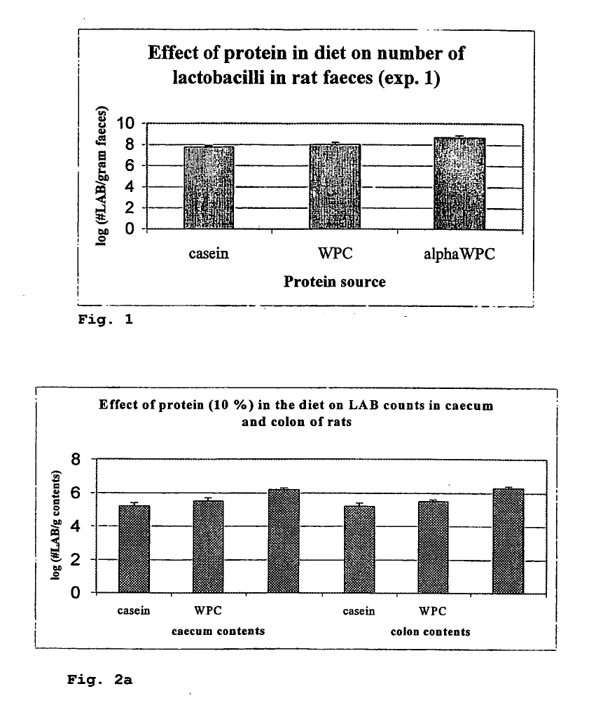

The results are shown in table 2 and in FIG. 1.

TABLE 2
Total numbers of lactobacilli in faeces of rats fed diets
containing different kinds of protein.

| Protein | Log10(lactobacilli/g | |
|---|---|---|
| source | faeces) | SD |
| Casein | 7.76 | 0.14 |
| WPC | 8.01 | 0.20 |
| αWPC | 8.68 | 0.19 |

As is shown in table 2 and FIG. 1, the number of lactobacilli in rat faeces is significantly increased when feeding an αWPC diet, compared to a casein or WPC diet. α-Lactalbumin contains a high amount of cystein, an important sulphur-containing amino acid. Without being bound to any explanation, it is believed that the prebiotic effect of αWPC may be due to one or more of the following aspects:

Taurin is a breakdown product of cystein and is, like glycin, a substrate for bile acid conjugation. Glycin-conjugated bile acids are more easily deconjugated than taurin-conjugated bile acids. Since deconjugated bile acids are toxic to lactic acid bacteria, a higher amount of cystein in the diet could lead to more taurin-conjugated bile acids, which is in favour of the lactic acid bacteria population.

Mucins are sulphur-containing substances, secreted by intestinal epithelial cells. When mucins no longer adhere to the intestinal epithelium, they are a substrate to lactic acid bacteria in the intestinal lumen. For this reason, a higher amount of cystein in the diet could lead to increased mucin production, which favours the growth of lactic acid bacteria.

Cystein and glutathion, which is made from cystein, are strongly reducing agents. A reducing environment promotes the survival of lactic acid bacteria.

### Example 2

Effect of Protein in the Diet on the Number of Lactobacilli in the Caecum and Colon of the Rat.

This experiment was performed as experiment 1. Rats were killed and the caecum was removed. Diets containing 10 or 20% protein were used. A diet containing 20% protein more closely resembles a human, western diet. In this case, part of the glucose in the diet was replaced by extra protein. In earlier experiments, only marginal diets containing 10% protein were used to increase the chance of finding significant effects.

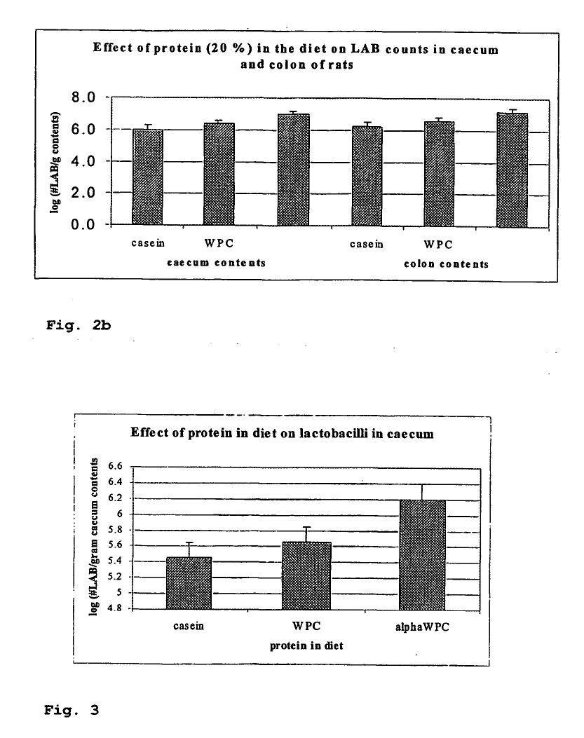

After the 14 day feeding period, the rats were killed and caecum and colon contents removed. Serial ten-fold dilutions of caecum and colon contents in physiological salt solution were plated onto MRS plates to determine the total Lactobacillus counts. The results are shown in table 3 and FIGS. 2A and 2B. The number of lactobacilli is significantly higher when feeding rats a diet containing αWPC compared to casein or WPC. The number of lactobacilli is significantly higher in both caecum and colon, and in both the 10% and 20% protein group when using αWPC instead of casein or WPC.

TABLE 3
Effect of 10 and 20% (w/w) of different kinds of protein
in the rat diet on the number of lactobacilli in
caecum and colon

| amount | | | | |
|---|---|---|---|---|
| of | | protein | | |
| protein | location | source | log(LAB/g) | SD |
| 10% | caecum | casein | 5.2 | 0.2 |
| | | WPC | 5.5 | 0.2 |
| | | alphaWPC | 6.2 | 0.1 |
| | colon | casein | 5.2 | 0.2 |
| | | WPC | 5.5 | 0.1 |
| | | alphaWPC | 6.3 | 0.1 |
| 20% | caecum | casein | 6.0 | 0.3 |
| | | WPC | 6.4 | 0.2 |
| | | alphaWPC | 7.0 | 0.2 |
| | colon | casein | 6.2 | 0.3 |
| | | WPC | 6.6 | 0.2 |
| | | alphaWPC | 7.2 | 0.2 |

### Example 3

Effect of Protein in the Rat Diet on the Total Count and Composition of the Lactobacillus Population in the Caecum.

The same rats as used in experiment 1 were used to determine the total number of lactobacilli in the caecum and to analyse the composition of the lactobacillus flora. At the end of the experiment, the rats were killed and the caecum was removed. The caecum content was diluted in a physiological salt solution by serial ten-fold dilution steps. Dilutions were plated onto MRS plates. The composition of the lactobacillus flora was determined by analysing a representative sample of the colonies on the MRS plates by RAPD-PCR, followed by 16S RNA sequence analysis.

The results are shown in table 4 and FIG. 3. As shown in table 4 and FIG. 3, the number of lactobacilli in the rat caecum is significantly higher when using an αWPC diet as compared to a casein or WPC diet. Moreover, the lactobacillus flora is more diverse as well, which is generally accepted as beneficial. αWPC promotes the growth of Lactobacillus reuteri and Lactobacillus acidophilus.

TABLE 4
Effect of different kinds of protein in the rat diet on the
number of lactobacilli and the composition of this flora in the
caecum.

| | # LAB in | # rats | | | |
|---|---|---|---|---|---|
| | caecum | containing | | | |
| Protein | (log | <100% | L. | L. | L. |
| source | cfu/g) | murinis | murinis | reuteri | acidophilus |
| casein | 5.46 ± | 2 (of 7) | 98 ± 1% | 2 ± 1% | 0 |
| | 0.19 | | | | |
| WPC | 5.66 ± | 2 (of 8) | 96 ± 3% | 4 ± 3% | 0 |
| | 0.19 | | | | |
| αWPC | 6.20 ± | 4 (of 8) | 87 ± 5% | 9 ± 5% | 4 ± 4% |
| | 0.20 | | | | |

### Example 4

Growth of Lactobacilli in Faecal Extracts.

Growth of lactobacilli in 10% (w/v) faecal extracts in water was monitored. The extracts were inoculated with L. acidophilus or L. reuteri, which were isolated from colonies obtained in experiment 3. By doing this, we used Lactobacillus species isolated from rats and characterised by 16S RNA analysis. The extracts, inoculated with lactobacilli, were incubated at 37° C., under anaerobic conditions. Growth was monitored by plating serial ten-fold dilutions onto MRS plates.

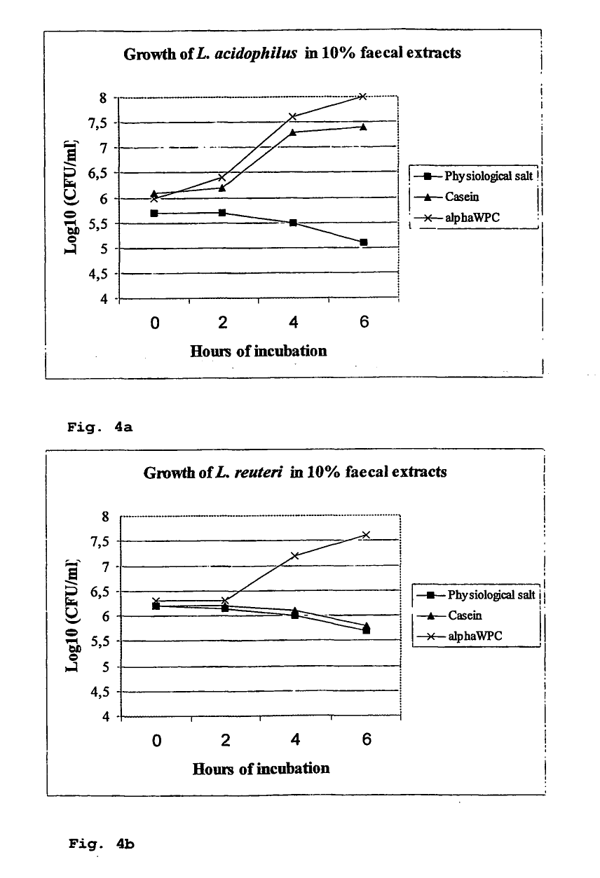

The results are shown in FIGS. 4a and 4b. FIG. 4a shows us that faecal extracts from rats consuming αWPC or casein diets support growth of Lactobacillus acidophilus to the same extent. Lactobacillus reuteri, however, is stimulated much more by faecal extracts from rats consuming αWPC as compared to casein. A diet rich in sulphur-containing amino acids (such as the αWPC diet) is expected to stimulate growth of lactobacilli, as explained earlier. In the case of growth in faecal extracts, the positive effect of increased mucin secretion on lactobacillus growth is believed to be the most profound. Mucins support growth of lactobacilli and can be detected in faeces. The found results correspond with the results of experiment 3, where αWPC also was found to stimulate L. reuteri and L. acidophilus.

### Example 5

Effect of αWPC on the Resistance Against Salmonella

Male Wistar rats, SPF, six weeks old, were divided into groups as depicted in table 5.

TABLE 5
Experimental set-up experiment 5.

| Group | Rat numbers | Protein source |
|---|---|---|
| A | 1, 3, 5, 7, 9, 11, | Casein |
| | 13, 15 | |
| B | 2, 4, 6, 8, 10, 12, | αWPC |
| | 14, 16 | |

The rats were fed semi-synthetic diets, differing in protein source as indicated in table 5. After 14 days, the animals were orally given 1 ml of a suspension containing 5*10⁸ CFU Salmonella enteritidis in a physiological salt solution. The course of the infection was followed by plating dilutions of fresh faeces onto Rambach agar plates (see: A. Rambach, Appl. Environm. Microbiol., 1990; 56: 301-303, purchased from E. Merck Nederland B. V., Amsterdam, The Netherlands), a selective medium for Salmonella species.

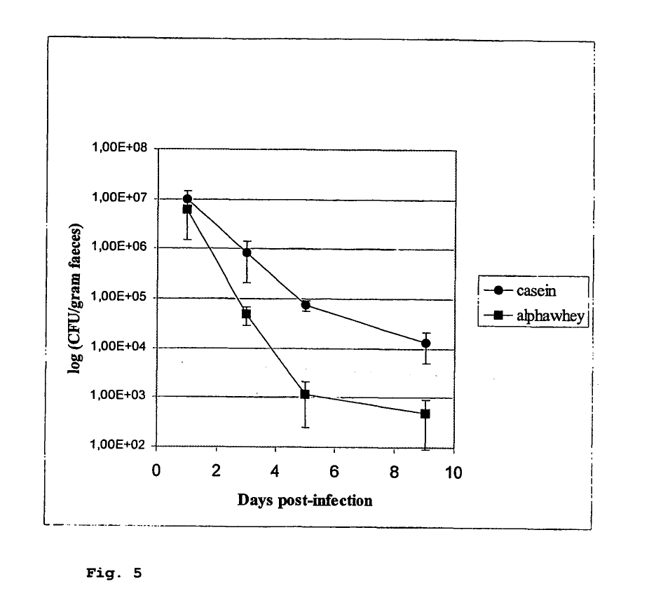

The results are shown in FIG. 5. This figure indicates that using αWPC in the diet leads to a significantly quicker wash out of salmonella. A fast wash out of salmonella means a quick recovery from disease. In other words, when no more salmonella is excreted, the infection is over. As said and shown before, a diet rich in α-lactalbumin (so, rich in cystein) will lead to increased numbers of lactic acid bacteria (LAB) in the gastro-intestinal tract. A strong LAB population will lead to increased colonisation resistance to e.g. Salmonella because of competition for adherence sites and nutrients and because of a lowering of pH.

1. Use of α-lactalbumin as a prebiotic food, food ingredient, food additive or food supplement.

2. Use of α-lactalbumin enriched whey protein concentrate as a prebiotic food, food ingredient, food additive or food supplement.

3. Use according to claim 2, wherein the α-lactalbumin content in the α-lactalbumin enriched whey protein concentrate is at least 60 w/w %.

4. Method for improving the prebiotic activity of a ready-to-consume food product, comprising the step of adding α-lactalbumin during the preparation of the said food product in such an amount that the α-lactalbumin content in the food product in ready-to-consume form is at least 0.5 w/w %, preferably 0.5-1.5 w/w %, more preferably about 1 w/w %.

5. Method according to claim 4, wherein the food product in ready-to-consume form is a dairy food product comprising casein, wherein the ratio α-lactalbumin:casein is at least 0.1, preferably between 0.1-0.6, more preferably between 0.2-0.4.

6. Method according to claim 4 or 5, wherein the food product in ready-to-consume form is a dairy product, preferably chosen from milk, fermented milk products, desserts, instant drinks, cheese and cheese products, preferably chosen from milk and fermented milk products, preferably yoghurt or a yoghurt product.

7. Method according to any of the preceding claims 4-6, wherein at least a portion of the α-lactalbumin is added as α-lactalbumin enriched whey protein concentrate.

8. Method according to any of the preceding claims 4-7, wherein the α-lactalbumin content in the α-lactalbumin enriched whey protein concentrate is at least 60 w/w %.

9. Method according to any of the preceding claims 4-8, further comprising the step of adding during the preparation of the said food product a food grade iron compound.

10. Ready-to-consume food product, comprising at least 0.5 w/w %, preferably 0.5-1.5 w/w %, more preferably about 1 w/w % α-lactalbumin.

11. Ready-to-consume food product according to claim 10, wherein the food product is a dairy product comprising casein, wherein the ratio α-lactalbumin:casein is at least 0.1, preferably between 0.1-0.6, more preferably between 0.2-0.4.

12. Ready-to-consume dairy food product according to claim 10 or 11, preferably chosen from milk, fermented milk products, desserts, instant drinks, cheese and cheese products, preferably chosen from milk and fermented milk products, preferably yoghurt or a yoghurt product.

13. Ready-to-consume dairy food product according to any of the claims 10-12, comprising a food grade iron compound.

14. Use of α-lactalbumin as active compound in the manufacture of a medicament for treatment of gastroenteritis.

15. Use of α-lactalbumin as active compound in the manufacture of a medicament for enhancing a resident population of lactic acid producing micro-organisms in the gastrointestinal tract of an animal.

16. Use according to any of the claims 14-15, wherein the α-lactalbumin content in the medicament is 0.2 w/w % or more, preferably 0.4 w/w % or more, most preferably 0.6 w/w % or more.

17. Use according to any of the claims 14-16, further comprising an iron compound.

18. Pharmaceutical composition, in particular for treatment of gastro-enteritis or for enhancing a resident population of lactic acid producing micro-organisms in the gastrointestinal tract of an animal, comprising as active ingredient 0.2 w/w % or more, preferably 0.4 w/w % or more, most preferably 0.6 w/w % or more α-lactalbumin.

19. Pharmaceutical composition, in particular for treatment of gastro-enteritis or for enhancing a resident population of lactic acid producing micro-organisms in the gastrointestinal tract of an animal, comprising α-lactalbumin and an iron compound as active ingredients.
